(19) United States
(12) Patent Application Publication
(10) Pub. No.: US 2009/0042840 A1
(43) Pub. Date: Feb. 12, 2009

(54) Phospholipids for Use in the Treatment of an Allergic Inflammatory Condition

(76) Inventors: Derek Alan Woodcock, Hertfordshire (GB); Christopher Pynn, Tadley (GB); Anthony Postle, South Hampton (GB)

Correspondence Address: NIXON & VANDERHYE, PC, 901 NORTH GLEBE ROAD, 11TH FLOOR, ARLINGTON, VA, 22203, US

(21) Appl. No.: 11/920,919

(22) PCT Filed: May 23, 2006
(86) PCT No.: PCT/GB2006/001885

§ 371 Date: Apr. 2, 2008

(30) Foreign Application Priority Data
May 23, 2005 (GB) 0510463.3

(51) Int. Cl. A61K 31/661; C07F 9/10

(52) U.S. Cl. 514/114; 558/172

(57) ABSTRACT

A phospholipid comprising a diacyl-substituted phosphatidyl group and a pharmaceutical composition comprising such a phospholipid for use in treatment of an allergic inflammatory condition and a method for the treatment of an allergic inflammatory condition. The phospholipid is particularly a mixture of phospholipids known as pumactant.

The present invention provides a phospholipid and a pharmaceutical composition for use in treatment of an allergic inflammatory condition and a method for treatment of an allergic inflammatory condition. The composition comprises a phospholipid, particularly a mixture of phospholipids known as pumactant.

There is a general need to find ways of improving the treatment of inflammation which is implicated in a wide variety of conditions.

According to the invention there is provided a phospholipid comprising a diacyl-substituted phosphatidyl group for use in the treatment of an allergic inflammatory condition.

Phospholipids naturally occur in the body, particularly at interfaces such as the surface of the lung where they provide a natural protective barrier which prevents irritants from stimulating receptors in the lung. It has been suggested that this natural barrier is deficient in asthmatics. A phospholipid such as pumactant has been disclosed for the treatment of asthma on the basis that it will restore the natural barrier, thereby reducing the number of receptors exposed to irritants. There has been no suggestion, though, that a phospholipid such as pumactant affects the tissue to which it is applied.

It has now been surprisingly found that a phospholipid such as pumactant does interact with the tissue to which it is applied such that it inhibits an anti-inflammatory response. The nature of this interaction is not known but its results can clearly be seen from Example 1 of the present application. From this Example, it can be seen that the phospholipid inhibits T-cell proliferation, especially lymphocyte secretion by T-cells. The data in Example 1 show the inhibition of T-cell proliferation in T-cells stimulated with ovalbumin peptide. Ovalbumin is an antigen commonly used to model allergic conditions such as allergic conjunctivitis and allergic airway disease, auto-immune conditions such as arthritis, and inflammation such as pulmonary inflammation. Accordingly it is clear that a phospholipid is useful in the treatment of such conditions.

According to the invention there is further provided a pharmaceutical composition for use in the treatment of an allergic inflammatory condition which composition comprises a phospholipid comprising a diacyl-substituted phosphatidyl group in association with a pharmaceutically acceptable diluent or carrier.

According to the invention there is also provided use of a phospholipid comprising a diacyl-substituted phosphatidyl group or of a pharmaceutical composition according to the invention in the manufacture of a medicament for use in the treatment of an allergic inflammatory condition.

Preferably, the phospholipid used according to the invention is a phospholipid according to formula (I):

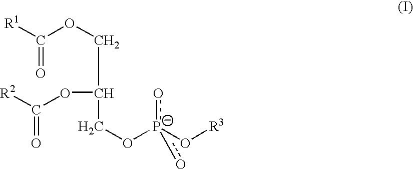

wherein R¹ and R² each independently represent a saturated or unsaturated alkyl group having from 13 to 21 carbon atoms; and
R³ represents a hydrogen atom or a choline, glycerol, ethanolamine, serine or inositol group; preferably R³ represents choline or glycerol.

Even more preferably, the phospholipid used in the invention is a phopsholipid according to formula (II):

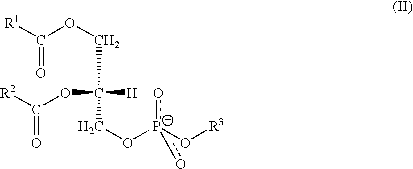

wherein R¹ and R² each independently represent a saturated or unsaturated alkyl group having from 13 to 21 carbon atoms; and
R³ represents a hydrogen atom or a choline, glycerol, ethanolamine, serine or inositol group; preferably R³ represents choline or glycerol.

The phospholipid for use in the invention has a phosphatidyl group substituted by two acyl groups. Preferably the phospholipid for use in the invention is a phosphatidyl group substituted by two acyl groups. The acyl groups may each comprise a saturated or unsaturated acyl radical generally having from 14 to 22 carbon atoms (such that R¹ and R² each independently represent a saturated or unsaturated alkyl group having from 13 to 21 carbon atoms), preferably from 16 to 20 carbon atoms (such that R¹ and R² preferably each independently represent a saturated or unsaturated alkyl group having from 15 to 19 carbon atoms).

Preferably the phospholipid may comprise by way of acyl radicals, the saturated radicals palmitoyl C16:0 (such that R¹ and R² preferably each independently represent a saturated alkyl group having 15 carbon atoms) and stearoyl C18:0 (such that R¹ and R² preferably each independently represent a saturated alkyl group having 17 carbon atoms) and/or the unsaturated radicals oleoyls C18:1 (such that R¹ and R² preferably each independently represent a mono-unsaturated alkyl group having 17 carbon atoms) and C18:2 (such that R¹ and R² preferably each independently represent a di-unsaturated alkyl group having 17 carbon atoms). The phospholipid more particularly comprises two identical saturated or unsaturated acyl radicals (such that R¹ and R² are preferably the same in that they represent identical alkyl groups), especially dipalmitoyl and distearoyl, or a mixture of phospholipids in which such radicals predominate, in particular mixtures in which dipalmitoyl is the major diacyl component.

The phospholipid is optionally either an animal-derived or a synthetic phospholipid; preferably it is a synthetic phospholipid.

An animal-derived phospholipid may be obtained in the usual way by mincing of or lavage from mammalian lungs, such as porcine or bovine lungs. Examples of animal-derived phospholipids which might be used include Curosurf (Chiesi Farmaceutici) which is produced from minced pig lungs and consists of 99% phospholipids and 1% surfactant proteins; Alveofact (Dr. Karl Thomae, Ltd., Germany) which is a compound obtained from bovine lung lavage and contains 90% phospholipids, about 1% proteins, 3% cholesterol, 0.5%o free fatty acids, and other components, including triglycerides; Survanta (Abbott, Ltd., Germany) which is prepared by lipid extraction of minced bovine lungs and contains approximately 84% phospholipids. 1% proteins, and 6% free fatty acids; BLES (BLES Biochemicals, Canada) which is produced by a bovine lung lavage; or Infasurf (Forest Labs) also known as calfactant which is produced by bovine calf lung lavage and contains 35 mg/ml phospholipids which are 26 mg/ml phosphatidyl choline (PC), 26 mg/ml dipalmitoylphosphatidylcholine (DPPC), 0.65 mg/ml protein and 0.26 mg/ml a hydrophobic peptide.

A synthetic phospholipid is preferably a diacyl phosphatidyl choline (DAPC) such as DPPC, dioleyl phosphatidyl choline (DOPC) or distearyl phosphatidyl choline (DSPC), phosphatidylglycerol (PG), PC, phosphatidylethanolamine (PE), phosphatidylserine (PS), phosphatidylinositol (PI), and/or phosphatidic acid.

The phospholipid is preferably a mixture of a diacyl phosphatidyl choline and a phosphatidyl glycerol. The phosphatidyl glycerol is advantageously a diacyl phosphatidyl glycerol. The acyl groups of the phosphatidyl glycerol, which may be the same or different, are advantageously each fatty acid acyl groups which may have from 14 to 22 carbon atoms. In practice, the phosphatidyl glycerol component may be a mixture of phosphatidyl glycerols containing different acyl groups. It is preferred for at least a proportion of the fatty acid acyl groups of the phosphatidyl glycerol to be unsaturated fatty acid residues, for example, mono-or di-unsaturated C18 (such that R¹ and R² preferably each independently represent a mono- or di-unsaturatecd alkyl group having 17 carbon atoms) or C20 fatty acid residues (such that R¹ and R² each independently represent a mono- or di-unsaturated alkyl group having 19 carbon atoms).

Preferred acyl substituents in the phosphaticlyl glycerol component are palmitoyl, oleoyl, linoleoyl, linolenoyl and arachidonoyl. Thus in the compounds of formula (I) or (II), when R³ represents glycerol, R¹ and R² each independently represent:
CH₃(CH₂)₁₄—;
CH₃(CH₂)₇CH═CH(CH₂)₇—;
CH₃(CH₂)₄CH═CHCH₂CH═CH(CH₂)₇—;
CH₃CH₂CH═CHCH₂CH═CHCH₂CH═CH(CH₂)₇—; or
CH₃(CH₂)₄[CH═CHCH₂]₄CH₂CH₂—. The phospholipid preferably comprises dipalmitoyl phosphatidyl choline and phosphatidyl glycerol.

The phospholipid is preferably a mixture of DPPC and PG. Even more preferably, the phospholipid is a mixture of DPPC and PG at a weight ratio of from 1:9 to 9:1, preferably from 6:4 to 8:2, more preferably about 7:3. DPPC can be prepared synthetically by acylation of glyceryl phosphoryl choline using the method of Baer & Bachrea, Can. J. of Biochem. Physiol 1959, 37, page 953 and is available commercially from Sigma (London) Ltd. PG may be prepared from egg phosphatidyl-choline by the methods of Comfurions et al, Biochem. Biophys Acta 1977, 488, pages 36 to 42; and Dawson, Biochem J. 1967, 102, pages 205 to 210, or from other phosphatidyl cholines, such as soy lecithin.

When co-precipitated with DPPC from a common solvent such as chloroform, PG forms with DPPC a fine powder. Preferably the phospholipid is a mixture of DPPC and a phosphatidyl glycerol derived from egg phosphatidyl choline, which results in phosphatidyl compounds substituted by a mixture of C16, C18 (saturated and unsaturated) and C20 (unsaturated) acyl groups. Such compounds are compounds of formula (I) or (II) wherein R¹ and R² each independently represent an alkyl group having 15 carbon atoms, a saturated or unsaturated alkyl group having 17 carbon atoms and an unsaturated alkyl group having 19 carbon atoms.

Examples of commercial synthetic phospholipid products include: Surfaxin (Discovery Labs) which is also known as lucinactant contains 26 molar parts of DPPC, 8 molar parts of POPG, 5 molar parts of PA and 1 part of KL-4; Lung Surfactant Factor LSF (Altana) which is also known as lusupultide contains recombinant SP-C, DPPC, PG and PA; Exosurf (GSK, Germany) which is composed of DPPC (˜84%), cetyl alcohol, and tyloxapol; or pumactant (Britannia Pharmaceuticals) which is composed of a mixture of DPPC and PG at a weight ratio of 7:3, may be used in the invention.

The phospholipid is preferably a phospholipid or a mixture of phospholipids which has a melting temperature which is about the same as or below body temperature (which is the temperature of the human or animal body to be treated). Such a mixture of phospholipids preferably contains a spreading phospholipid which has a melting temperature which is about the same as or below body temperature such as PG, PE, PS, or PI.

The phospholipid is preferably applied at a rate of from 1, preferably from 10, more preferably from 50 to 1000, preferably to 800, more preferably to 300 μg per square centimetre of inflamed area.

The phospholipid is preferably applied in the form of a dry powder. More generally, the powdered phospholipid may have a particle size in the range of 0.5 to 100 μm, more suitably of 0.5 to 20 μm, preferably 0.5 to 10 μm. The phospholipid is preferably a surface active phospholipid (SAPL).

The phospholipid or pharmaceutical composition according to the invention is preferably for use in the treatment of an inflammatory condition. A suitable inflammatory condition to be treated by the present invention is an inflamed wound, an auto-immune condition (such as arthritis), an allergic condition (such as seasonally affected asthma, perennial affected asthma, rhinitis, hay fever), and/or an allergic reaction (e.g. an insect bite).

The inflamed wound to be treated by the invention is preferably an opening or abrasion on a surface of a human or animal body not caused by surgery. The surface of a human or animal body to be treated is optionally either an internal or external surface.

The treatment of inflammation in the invention preferably comprises inhibiting lymphocyte secretion and/or T-cell proliferation.

The pharmaceutical composition according to the invention comprises a pharmaceutically acceptable excipient. Any compatible excipient may be used. The excipient is preferably free from water. Where the carrier or diluent is a liquid, it is preferably non-aqueous. The excipient preferably comprises a surface active agent. A surface active agent is useful because it enables a phospholipid having a melting temperature above body temperature to be used in the composition. More preferably the surface active agent is a pharmaceutically acceptable surfactant or hydrophobic protein. Examples of such agents include: KL-4 which is 21 amino acid synthetic peptide; tyloxapol which is a nonionic surfactant; cetyl alcohol (or hexadecanol); or cholesteryl palmitate. A further suitable excipient is a protein, especially a protein which improves absorption such as apoprotein B.

When the composition is provided in liquid form, the excipient preferably comprises a carrier liquid in which the phospholipid is dispersed or dissolved. The carrier liquid is typically one which is substantially non-volatile or only sparingly volatile at body temperature. A suitable carrier includes a physiologically acceptable glycol, especially a propylene glycol, polyethylene glycol and/or glycerol.

The composition may optionally be provided in liquid, semi-liquid or pasty form. Pastes can be prepared by simply dispersing a phospholipid in a suitable carrier, or, when appropriate, dissolving the phospholipid in a heated carrier and allowing the phospholipid to precipitate as a powder on cooling, preferably at a loading that will form a paste. Propylene glycol is especially effective as a carrier because at room temperature a phospholipid may be dispersed in it as a paste, but at body temperature a mobile solution is formed. Also polyethylene glycols may be prepared which are waxy solids at room temperature and liquids at body temperature, such as for example PEG 600. Various dispersions of a phospholipid in propylene glycol are described in U.S. Pat. No. 6,133,249, the entire contents of which are incorporated herein by reference.

According to the invention, there is further provided a method of treating inflammation which method comprises applying to a human or animal patient in need of such treatment a therapeutically effective amount of a phospholipid. The phospholipid is preferably in the form of a pharmaceutical composition according to the invention.

The invention is illustrated with reference to the following Figures of drawings which are not intended to limit the scope of the claims and in which:

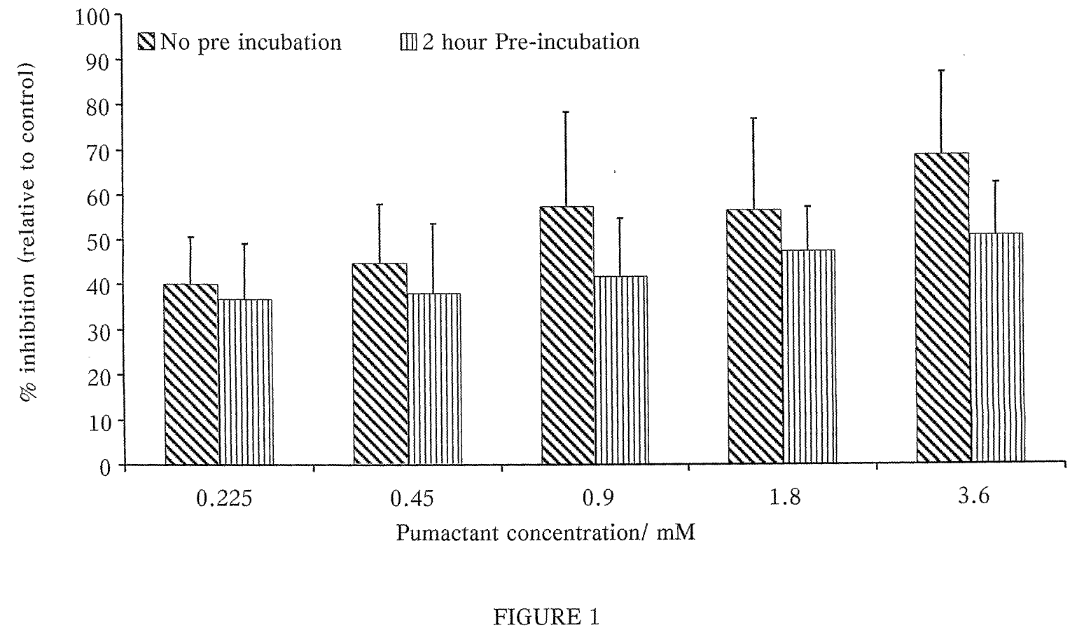

FIG. 1 is a graph comparing the percentage inhibition relative to control of T-cells either pre-treated by differing amounts of pumactant 2 hours before; and

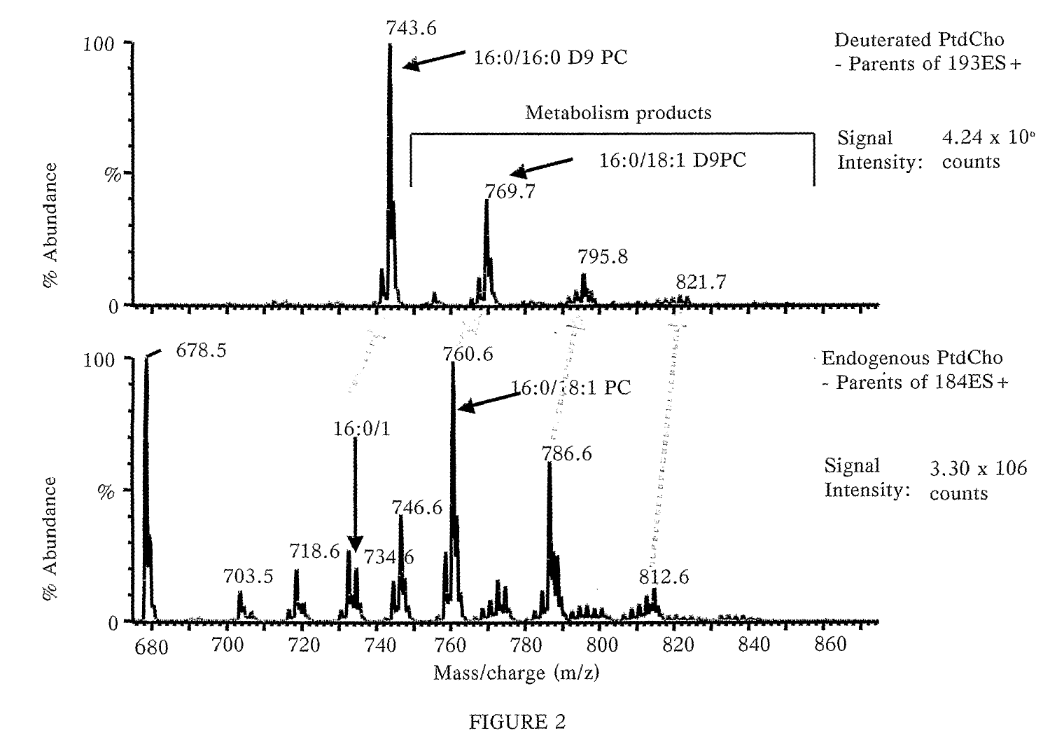

FIG. 2 is a ESI-MS mass-spectograph profile of B3Z phosphatidyl choline (referred to as PtdCho) following incubation with D-9 DPPC (referred to as Deuterated Ptd Cho) for 24 hours.

The invention will now be illustrated with reference to the following Examples which are not intended to limit the scope of the claims.

The experiments in the Examples were designed to evaluate the mechanism of interaction between Pumactant and immune cells of lymphocyte cell lineage. They demonstrate that, at concentrations causing greater than 50% inhibition of the cellular immune response, there is only a very limited alteration to bulk cellular phospholipid composition. The modification to membrane phospholipid composition caused by incubation with Pumactant is, nevertheless, highly reproducible. These results show that inhibition of cellular immune responses by Pumactant cannot be due to alterations to the composition of total membrane cell phospholipid but are instead mediated by a much more specific and targeted mechanism.

### EXAMPLE 1

Mouse T-cell hybridoma cells (B3Z cells, ovalbumin-specific with β-galactosidase (LacZ) NFAT-regulated reporter construct) were incubated with Pumactant (70% dipalmitylphosphatidylcholine (DPPC), 30% egg phosphatidylglycerol) for 0-2 hours at a concentration of 0-3.6 mM. Cells were then stimulated with an ovalbumin-derived peptide for 24 hours in the presence of the indicated concentration of Pumactant. Cell activation was measured by LacZ expression, monitored colorimetrically by the formation of the reaction products of the β-galactosidase enzyme.

The results are shown in FIG. 1. It can be seen that activation of B3Z cells was inhibited by 39% by incubation with 0.22 mM Pumactant. At 3.6 mM the inhibition reached 69%. The extent of this inhibitory response to Pumactant was not enhanced by pre-incubation for 2 hours before cell stimulation. This response demonstrates that the inhibitory response to Pumactant is rapid and does not require previous alteration to cell membrane phospholipid composition.

### EXAMPLE 2

To investigate the mechanism of interaction between Pumactant and lymphocytes, 10⁷ B3Z cells were incubated for up to 24 hours with a sterile emulsion of 3.6 mM Pumactant in which 7% (w:w) of the DPPC had been replaced with DPPC containing the stable isotope deuterium (methyl-d9)-choline (d9-DPPC). After incubation for 2, 6 or 24 hours, total cell lipids were extracted using chloroform and methanol and their phospholipid compositions quantified by electrospray ionisation mass spectrometry. Native phosphatidylcholine (PC) species were determined by precursor scans of m/z184, while PC species containing the (methyl-d9) label were determined by equivalent scans of m/z 193.

In control B3Z cells, DPPC was a very minor component. Incubation with Pumactant for 2 hours increased the cellular content of DPPC by 280%, but the absolute concentration of DPPC never exceeded 10% of cellular PC. After incubating growing cells for 24 hours, cell number and total membrane phospholipid concentration both doubled. Even under these conditions, however, the total cellular DPPC was never greater than the 10% observed at 2 hours. These results demonstrate that uptake of Pumactant was limited and could be readily saturated, even when Pumactant was present continuously in a 20-fold concentration excess (calculated as the concentration ratio of added Pumactant to measured cell PC concentrations).

The direct incorporation of Pumactant into B3Z cell PC was demonstrated by following the fate of the d9-DPPC component in the modified Pumactant. Results of these analyses demonstrated that by 24 hours, over 50% of the Pumactant PC taken up by immune cells was still present as DPPC, with the remainder being converted to more unsaturated PC molecules. These results are illustrated in FIG. 2.

1. A phospholipid comprising a diacyl-substituted phosphatidyl group for use in the treatment of an allergic inflammatory condition.

2. A phospholipid as defined in claim 1 according to formula (I): 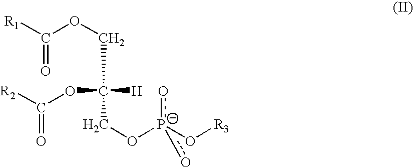
wherein R¹ and R² each independently represent a saturated or unsaturated alkyl group having from 13 to 21 carbon atoms; preferably R¹ and R² each independently represent a saturated or unsaturated alkyl group having from 15 to 19 carbon atoms; and
R³ represents a hydrogen atom or a choline, glycerol, ethanolamine, serine or inositol group; preferably R³ represents choline or glycerol.

3. A phospholipid as defined in claim 2 according to formula (II): 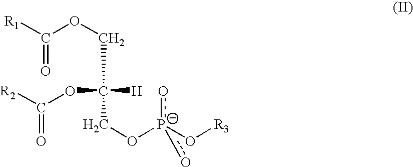
wherein R¹, R² and R³ are each as defined in claim 2.

4. A phospholipid as defined in claim 2 wherein R³ represents glycerol and R¹ and R² each independently represent:
CH₃(CH₂)₁₄—;
CH₃(CH₂)₇CH═CH(CH₂)₇—;
CH₃(CH₂)₄CH═CHCH₂CH═CH(CH₂)₇—;
CH₃CH₂CH═CHCH₂CH═CHCH2CH═CH(CH₂)₇—; or
CH₃(CH₂)₄[CH═CHCH₂]₄CH₂CH₂—.

5. A phospholipid as defined in claim 1 which is a phosphatidyl choline, phosphatidyl glycerol, phosphatidyl ethanolamine, phosphatidyl serine, phosphatidyl inositol, and/or a phosphatidic acid.

6. A phospholipid as defined claim 1 which is a mixture of dipalmitoylphosphatidylcholine and phosphatidylglycerol at a weight ratio of from 1:9 to 9:1, preferably from 6:4 to 8:2, more preferably about 7:3.

7. A phospholipid as defined in claim 1 which is a synthetic phospholipid.

8. A phospholipid as defined in claim 1 which is used at a rate of from 1, preferably from 10, more preferably from 50 to 1000, preferably to 800, more preferably to 300 μg per square centimetre of inflammation.

9. A phospholipid as defined in claim 1 which is in the form of a dry powder.

10. A phospholipid as defined in claim 1 wherein the treatment comprises inhibiting lymphocyte secretion.

11. A pharmaceutical composition for use in the treatment of an allergic inflammatory condition which composition comprises a phospholipid comprising a diacyl-substituted phosphatidyl group in association with a pharmaceutically acceptable excipient.

12. A composition as defined in claim 11 wherein the phospholipid is a phospholipid of formula (I): 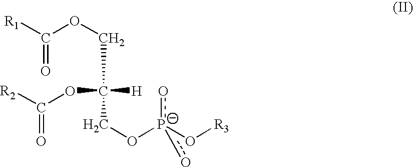
wherein R¹ and R² each independently represent a saturated or unsaturated alkyl group having from 13 to 21 carbon atoms, preferably R¹ and R² each independently represent a saturated or unsaturated alkyl group having from 15 to 19 carbon atoms; and
R³ represents a hydrogen atom or a choline, gycerol, ethanolamine, serine or inositol group; preferably R³ represents choline or glycerol.

13. A composition as defined in claim 11 wherein the treatment comprises inhibiting lymphocyte secretion.

14. A composition as defined in claim 11 wherein the excipient is a surface active agent, a protein, and/or a carrier liquid.

15. A medicament for use in the treatment of an allergic inflammatory condition which comprises a phospholipid comprising a diacyl-substituted phosphatidyl group in association with a pharmaceutically acceptable excipient.

16. A medicament as defined in claim 15 for inhibiting lymphocyte secretion.

17. A method of treating an allergic inflammatory condition which method comprises applying to a human or animal patient in need of such treatment a therapeutically effective amount of a phospholipid comprising a diacyl-substituted phosphatidyl group.

18. A method as defined in claim 17 wherein the phospholipid is a phospholipids of formula (I): 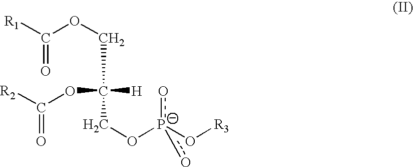
wherein R¹ and R² each independently represent a saturated or unsaturated alkyl group having from 13 to 21 carbon atoms; preferably R¹ and R² each independently represent a saturated or unsaturated alkyl group having from 15 to 19 carbon atoms; and
R³ represents a hydrogen atom or a choline, glycerol, ethanolamine, serine or inositol group; preferably R³ represents choline or glycerol.

19. A method as defined in claim 17 wherein the phospholipid is in the form of a pharmaceutical composition for use in the treatment of an allergic inflammatory condition which composition comprises a phospholipid comprising a diacyl-substituted phosphatidyl group in association with a pharmaceutically acceptable excipient.

20. A method as defined in claim 17 wherein the phospholipid inhibits lymphocyte secretion.
